(19) United States
(12) Patent Application Publication
(10) Pub. No.: US 2011/0171083 A1
(43) Pub. Date: Jul. 14, 2011

(54) Pneumatic hand dryer with integrated sanitizer

(76) Inventor: Julian Paul Swistak, Westfield, MA (US)

(21) Appl. No.: 12/614,442

(22) Filed: Jan. 11, 2010

(51) Int. Cl. A47K 10/48; F26B 21/06; F26B 21/00; A61L 2/16

(52) U.S. Cl. 422/292; 34/562; 34/72

(57) ABSTRACT

This is a push-button-timer activated pneumatic device for removing water moisture from hands. Integrated ahead of the flow of air discharged from the hand dryer is a pressure regulator and lubricator that shall contain a sanitizing solution that is delivered in a parts-per-million ratio out through the diffused nozzle. This system can have all of the components is one unit, or can be installed with a central regulator/lubricator-sanitizer and an array of satellite hand dryers. This system is obtains pressurized air from a central compressor and is ideal for use in locations that typically use compressed air, such as manufacturing facilities.

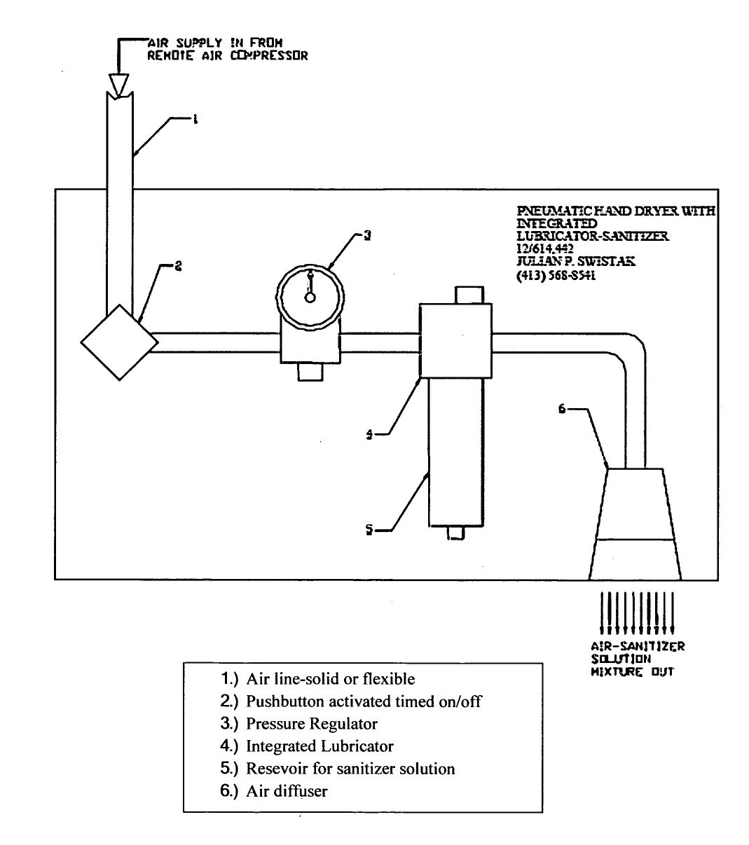

The inventor is Julian P. Swistak, citizen of the USA. Julian P. Swistak is the sole inventor of the Pneumatic Hand Dryer with Integrated Lubricator-Sanitizer and he resides in Westfield Mass.

The pneumatic hand dryer is an invention intended to introduce the ability to use compressed air to dry hands by integrating a sanitizing solution delivery system to limit the transmission of germs. This pneumatic hand dryer is an ideal device for use in washrooms where a supply of compressed air is readily available because it uses no electricity to function; all that is required to make the device functional is a supply of compressed air.

### BACKGROUND OF THE INVENTION

The pneumatic hand dryer is an inexpensive way to reduce the use of paper hand towels and limit the transfer of germs. The idea for the use of a sanitizing solution lubricator originated in the modern machine shop and in response to the H1N1 flu threat. Compressed air is used to operate machine tools and is a pressure regulator and lubricator is needed to introduce oil as an anti-corrosive throughout the machine's pneumatic system. With the pneumatic hand dryer the lubricator holds a sanitizing solution instead of oil; the hands are dried and sanitized by the air blast discharged from the dryer.

The idea for the pneumatic hand dryer/sanitizer is in response for the need for manufacturing facilities to achieve zero-waste status by reducing the trash resulting from paper hand towels. The byproduct of having a compressed air supply is the ability to use this device to dry hands without waste-paper and to reduce the spread of germs because of the integrated pneumatic sanitizer/lubricator.

The description of the drawing is as follows: A one dimensional view has been provided and is approximately one-half scale. The drawing depicts the six basic parts attached to a mounting plate. The six basic parts make this a working pneumatic hand dryer with integrated lubricator-sanitizer. Part number 1 is the rigid or flexible air line that brings in a supply of compressed air to the invention. Rigid or flexible air line connects the details of the hand dryer to create a linear flow through the parts. Part number 2 is a pushbutton or movement-activated on-off switch to activate the flow of compressed air through the hand dryer. Part number 3 is an air pressure regulator to act as a safety device to control the pressure of the discharge of air-sanitizer mixture. The regulator also helps to meter the concentration of sanitizer mixed with the compressed air. Part number 4 is the in-line integrated Lubricator. This commercially available device normally introduces petroleum or synthetic oil into compressed air systems but this invention changes the purpose of the lubricator from an oil mist device to sanitizing solution mist device. Part 5 is the reservoir for the sanitizing solution. (Note: Parts 3, 4 and 5 can be located remotely and used to supply a series of pneumatic hand dryers). Part 6 is an air diffuser that delivers the flow of air-sanitizer mixture out of the hand dryer to dry and sanitize wet hands efficiently. A decorative and functional cover would be used to protect and secure the 6 details of the invention, but is not shown in the drawing because it is not critical to the functionality of the invention.

This is a substitute specification for an Incomplete Non-provisional Application for application Ser. No. 12/614,442 and this substitute document contains no new material.

What is claimed is:

1. A pneumatic hand drying device that is different from the electric-powered hand dryer because of the ability to introduce a sanitizing solution into the air blast used to dry the hands. Said hand dryer uses no electricity or electrical wiring.

2. The ability to utilize a central sanitizing lubricator that deploys the sanitizing solution to an array of said pneumatic hand dryers. The hand said hand dryer shall be wall mounted but the said central sanitizing lubricator may be hidden for utility or service requirements.

3. The said hand dryers shall be tamperproof with an air-pressure regulator and diffused nozzle to safely discharge the air mixture in accordance with OSHA safety regulations.

4. Said hand dryers shall be constructed with intent to be serviced, inspected and said sanitizing solution lubricator has the ability to be adjusted for air/said sanitizer solution mixture ratio. Said sanitizing lubricator shall be refillable.

5. This said refilling feature will allow said sanitizing solution to be modified for future health threats or improved sanitizing solution products.

6. Said pneumatic hand dryer shall have a pneumatic push button that activates a timed blast of air deemed sufficient enough to dry hands and introduce said sanitizing solution.
